(19) United States
(12) Patent Application Publication
(10) Pub. No.: US 2026/0286276 A1
(43) Pub. Date: Sep. 24, 2026

(54) METHODS AND SYSTEMS FOR AUTOMATED MEDIA SUPPLY AND CONTROL

(71) Applicant: Molecular Devices (Austria) GmbH, Puch Bei Hallein (AT)

(72) Inventors: Markus AICHER, Puch Bei Hallein (AT); Steven KENNEDY, Puch Bei Hallein (AT); Felix SPIRA, Puch Bei Hallein (AT); Andreas GFROERER, Puch Bei Hallein (AT); Josef ATZLER, Puch Bei Hallein (AT)

(73) Assignee: Molecular Devices (Austria) GmbH, Puch Bei Hallein (AT)

(21) Appl. No.: 19/469,424

(22) PCT Filed: Apr. 17, 2024
(86) PCT No.: PCT/IB2024/053762

§ 371 Date: Sep. 26, 2025

Related U.S. Application Data
(60) Provisional application No. 63/496,560, filed on Apr. 17, 2023.

(51) Int. Cl. C12M 1/34; C12M 1/00; C12M 1/12; C12M 1/36; C12M 3/00

(52) U.S. Cl. CPC C12M 41/44; C12M 23/22; C12M 23/38; C12M 23/48; C12M 37/04; C12M 41/12; C12M 41/48

(57) ABSTRACT

A cell culture media container includes a body including a body including a base and a plurality of walls extending from the base, wherein the plurality of walls together define a container opening, a sealing lid removably coupled to the body, the sealing lid including a removable access lid defined in an access opening of the sealing lid, the removable access lid being configured to be removed from the sealing lid, and a seal disposed about a periphery of the sealing lid, wherein the seal is configured to removably secure the sealing lid to the container opening, wherein an interior floor of the base has two slanted floor portions, the slanted floor portions intersecting along a line located under the access opening in a direction perpendicular to a surface of the sealing lid.

### CROSS-REFERENCE TO RELATED APPLICATION

This application claims the benefit of U.S. Provisional Application No. 63/496,560, filed on Apr. 17, 2023, the disclosure of which is hereby incorporated by reference in its entirety.

### BACKGROUND

Manual cell culturing typically requires controlling media and reagent temperatures, mixing media and reagents as needed, and performing operations to handle the reagents and media. These are historically manual processes performed by technicians using discrete pieces of tabletop equipment as needed during a cell culturing process.

### SUMMARY

In general terms, this disclosure is directed to methods and systems for automated media supply and control. In one aspect, the technology relates to a cell culture media container including a body including a body including a base and a plurality of walls extending from the base, wherein the plurality of walls together define a container opening, a sealing lid removably coupled to the body, the sealing lid including a removable access lid defined in an access opening of the sealing lid, the removable access lid being configured to be removed from the sealing lid, and a seal disposed about a periphery of the sealing lid, wherein the seal is configured to removably secure the sealing lid to the container opening, wherein an interior floor of the base has two slanted floor portions, the slanted floor portions intersecting along a line located under the access opening in a direction perpendicular to a surface of the sealing lid.

In an example of the above aspect, the body further includes an insulating material. In another example, the insulating material is disposed about the plurality of walls, defines a lateral outermost extent of the body and covers an outer surface of the plurality of walls. In a further example, the base of the body is exposed. In yet another example, the insulating material includes one of polystyrene and polyurethane. In various examples, the sealing lid is configured to be removed from the container opening in the direction perpendicular to a surface thereof, and the removable access lid is configured to be removed in the direction perpendicular to the surface of the sealing lid therefrom at the access opening. For example, a first removal force to remove the sealing lid from the body is greater than a second removal force to remove the removable access lid from the sealing lid. In an example, the seal includes an O-ring gasket. In a further example, the base includes a first slanted portion pitched at a first angle to a plane defined by a first wall of the plurality of walls. In yet another example, the base further includes a second slanted portion pitched at a second angle to the plane defined by the first wall of the plurality of walls. In further examples, the first angle is different than the second angle. For example, the line defines a lowermost depth of the media container.

In other examples, the access opening is defined by an elongate access opening axis. In a further example, the line is substantially parallel to the elongate access opening axis. In yet another example, the body includes an RFID sensor attached thereto. In other examples, the sealing lid includes a detent configured to receive the removable access lid. In further example, the body defines an opening, the opening including a rim and the sealing lid has a flange configured to snugly couple with the rim. For example, the body includes one of metal, plastic, or a combination thereof. In additional examples, the plurality of walls are opaque to external light. In further examples, the seal includes a penetrable material. In additional examples, the penetrable material includes a foil. In an additional example, the body includes one of a media trough, an adapter block for centrifuge tubes, and a vessel configure to hold a media volume corresponding to a volume of one or more pipettes.

In another aspect, the technology relates to an automated media handling system including a tray configured to arrange one or more cell culture media containers thereon, one or more stations underneath the tray, each of the one or more stations including, a heating element disposed below the tray and thermally coupled to the one or more cell culture media containers arranged thereon, a cooling element disposed below the tray and thermally coupled to the one or more cell culture media containers arranged thereon, and a magnetic motor configured to rotatably actuate a stirring element held in one or more of the cell culture media containers arranged thereon, a robotic arrangement configured to manipulate the cell culture media containers, and a controller configured to control operation of the cell culture media containers and of the one or more stations.

In an example of the above aspect, a surface of the tray is at an angle to a horizontal plane. In another example, the one or more stations include a plurality of container stations, and the tray further includes a channel disposed between adjacent container stations. In a further example, the channel is disposed adjacent an upper-most edge of each container station. In yet another example, the system further includes a moat disposed adjacent a lower-most edge of each of container station. In other examples, the system further includes a drain fluidically coupled to the moat. For example, the heating element includes an electric heating element. In other examples, the cooling element includes a refrigerant cooling loop. In yet another example, at least one of the heating element and the cooling element include a hydronic channel. In further examples, the heating element includes the cooling element. In a further example, the tray defines a perimeter wall at least partially surrounding the one or more stations. For example, the perimeter wall defines an opening associated with each of the plurality of stations. In other examples, the heating element and the cooling element of one or more stations are individually controllable. In additional examples, the heating element is disposed between the tray and the magnetic motor. In further examples, the one or more stations includes up to eight container stations. In another example, adjacent media containers have slanted floor surfaces that are pitched away from each other. In an example, every other media container arranged on the tray has a floor surface that are pitched in a same direction.

### BRIEF DESCRIPTION OF THE DRAWINGS

FIGS. 1A-1B illustrate partial exterior views of a cell culturing system, in accordance with various examples of the disclosure.

FIGS. 2A-2H are illustrations of an automated media handling subsystem of an automated cell culturing system, in accordance with various examples of the disclosure.

FIGS. 3A and 3B are side views of a media trough, according to various examples of the disclosure.

FIGS. 4A-4D illustrate perspective and exploded views of a media trough, in accordance with examples of the disclosure.

FIGS. 5A and 5B are cross-sections of a media trough system, in accordance with various examples of the disclosure.

FIG. 6 illustrates a supply apparatus to an automated cell culturing system, in accordance with various examples of the disclosure.

FIG. 7 depicts a block diagram of a computing device configured to control an automated media trough device, according to various examples of the disclosure.

### DETAILED DESCRIPTION

Various embodiments are described herein in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

Examples of the disclosure include vessels for storing media in an automated cell culturing system. These vessels are referred to herein as “media troughs,” and may hold any type of media or reagent typically used in cell culturing. Such media include, for example, medias and dyes used in cell culturing, assays, organoid and spheroid formation and cultivation, stem cells cultivation, such as, e.g., induced pluripotent stem cell media, induction media, aggregation media, mesoderm specification media, cardiomyocytes differentiation media, maintenance media, mid-brain organoids, dyes, antibodies, and the like. The troughs may also include reagents such as, e.g., phosphate buffered saline, trypsin, or the like. The automated cell culturing system may access these media or reagents as required for a particular cell culturing process, and in the automated cell culturing system, the media troughs have certain structural features that enable them to be incorporated into an automated system. Accordingly, it may be advantageous to have automated and efficient processing of media troughs to mix media and reagents in and out of various troughs configured to hold media and reagents therein.

Examples of the disclosure relate to an automated supply of media and reagents. In particular, examples of the disclosure relate to a method and system for automated media and reagent supply. Examples of the disclosure include an integrated and automated reagent and media supply system, the system including storage and preconditioning arrangements for reagents and media present in the various vessels, or throughs, of the system. Integrated functionalities of the system include various operations such as, e.g., cooling, heating, mixing and pre-conditioning of the reagents and media. In various examples, the reagent and media supply system may include an insulative and protective structure to minimize the effects of deleterious factors such as, e.g., condensing water on walls of the troughs and allowing precise temperature control of the media or liquid held in the media troughs. The insulating material may be, e.g., a removable insulating material. In examples, the media troughs are configured for automated liquid handling by being coupled to a controller, e.g., to minimize any residual volume, and include a lid to avoid, prevent or reduce evaporation. In order to improve process-reliability, radio frequency identification (RFID)-tags may be integrated into the vessels or troughs, and may also be connected to the controller. Accordingly, the system according to various examples includes highly integrated and dedicated functionalities such as cooling, heating, mixing, prevention of condensation, prevention of contamination, minimized residual volume, and minimized evaporation, as well as automatic vessel and reagent identification via, e.g., RFID, controller via a controller.

In various examples, the system also includes higher process stability and reproducibility, less contamination, and improved reagent stability. The example system also allows handling of a number of different media, as well as the automated addition of reagents to a media. The example system further provides better process stability and reproducibility compared to conventional systems, which provides a user with a higher convenience.

Another technical problem is the need for storing different types of media and/or reagents for varying lengths of time at varying required temperatures. Examples of the present disclosure provide a technical solution to the above technical problem by allowing to solve the need for storing different types of media on the system for many days, and allows to have, e.g., the correct media temperature when required. Accordingly, a user is able to automatically perform tasks such as, e.g., stem cell passaging, over a long period of time such as, e.g., a few days. Accordingly, the system reduces the hands-on time for a user, and the RFID tags or chips, also referred to herein as RFID sensors, coupled to a controller and that can be read by an RFID antenna, may reduce the possibility of errors such as, e.g., using the wrong media and/or reagent.

Various examples of the disclosure include example trays that support media troughs in an automated cell culturing system, the media troughs being configured to hold media and/or reagent. The media troughs themselves may hold various types of media and/or reagents typically utilized in cell culturing, and have structural features that enable them to be incorporated into an automated system. For example, the trays that support the media troughs include heating or cooling components to heat or cool the troughs, can activate a mixer within the troughs, and can drain condensate away from the troughs. These example trays enable automation of the cell culturing processes.

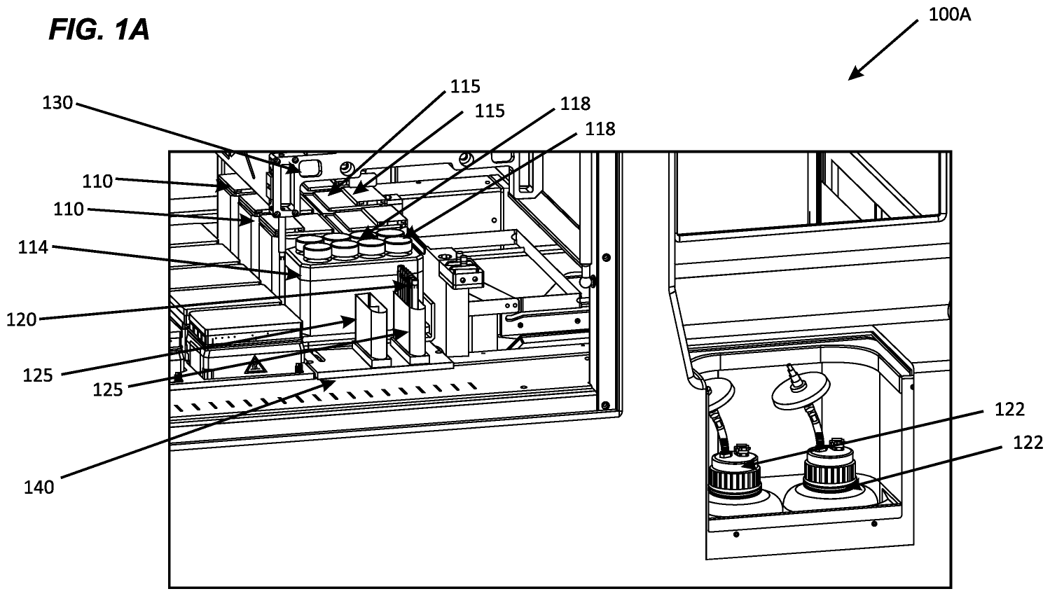

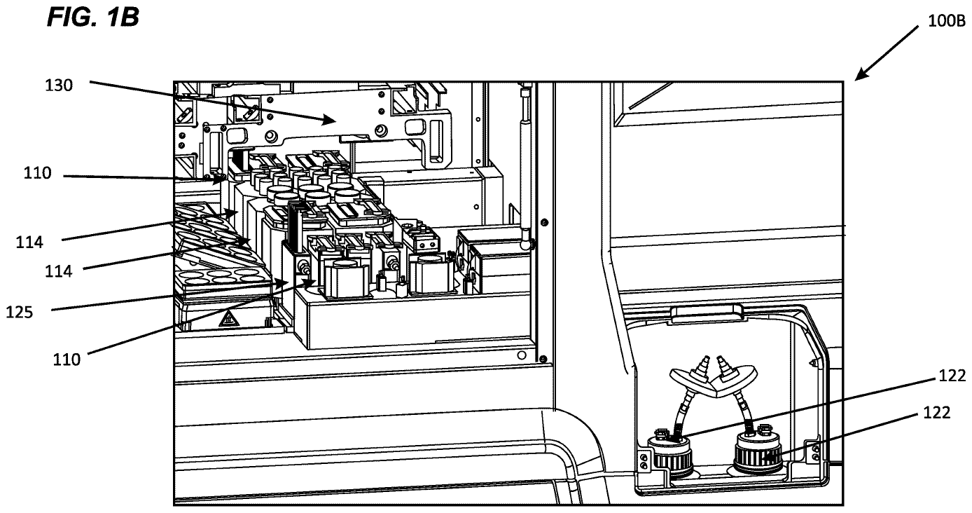

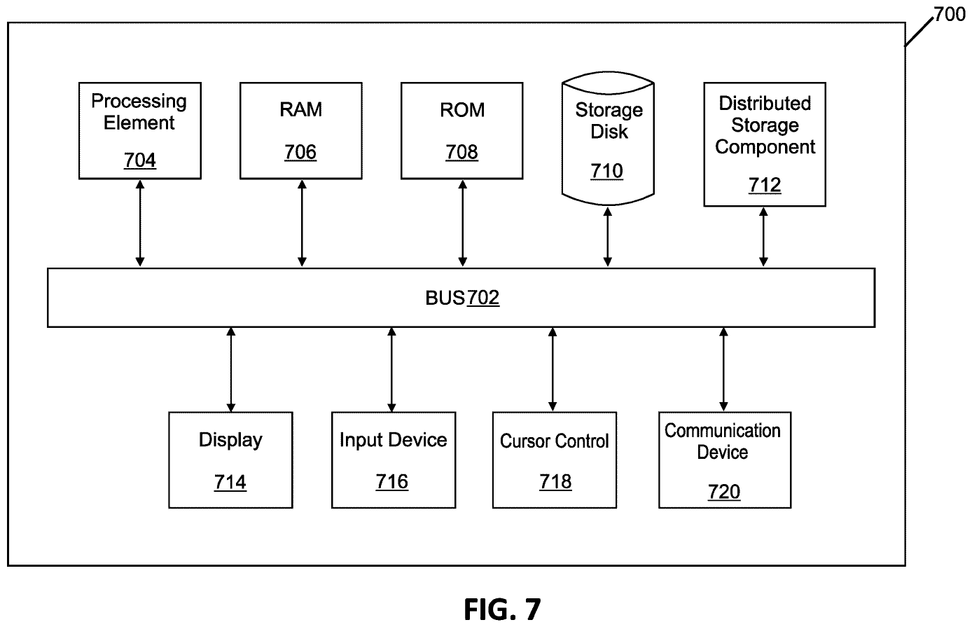

FIGS. 1A-1B illustrate partial exterior views of a cell culturing system, in accordance with various examples of the disclosure. In FIG. 1A, the automated system 100A includes a plurality of media troughs 110, centrifuge tube adapters 114 and small vessels 125, that are closed by lids 115 or 118. Some of the lids 115 and 118 are sealing lids, and some of the lids 115 and 118 are removable access lids, as further discussed below. The access lids 115 may allow e.g., pipettes, to be inserted in and out of the media troughs 110 to add or remove media as required. In other examples, the automated cell culturing system 100A includes a robotic arrangement 130 configured to perform a number of operations such as, e.g., opening and closing the removable access lids 115 or 118, gripping and transporting the pipettes 120 from additional media trough or small vessel 125, and bringing the pipettes 120 into one or more of the media troughs 110 or small vessels 125 to, e.g., remove media and/or reagent therefrom and transport the removed media and/or reagent to a desired location. For example, the robotic arrangement 130 may be a discrete mechanism within the automated cell culture system 100A, or may be a separate robotic arrangement 130. In examples, the robotic arrangement 130 may be configured to, in sequence, open a removable access lid 115, transport the pipettes 120 to the media trough 110 or small vessel 125, draw an amount of media and/or reagent from the media trough 110 or small vessel 125 into the pipettes 120, transfer the pipettes 120, holding the drawn media and/or reagent therein, to a desired destination, and close the removable access lid 115 on the media trough 110 or small vessel 125. The robotic arrangement 130 may then bring the pipettes 120 back in the media trough 110 or small vessel 125. The robotic arrangement 130 may be controller via a computing device such as, e.g., the computing device 700 described below with respect to FIG. 7.

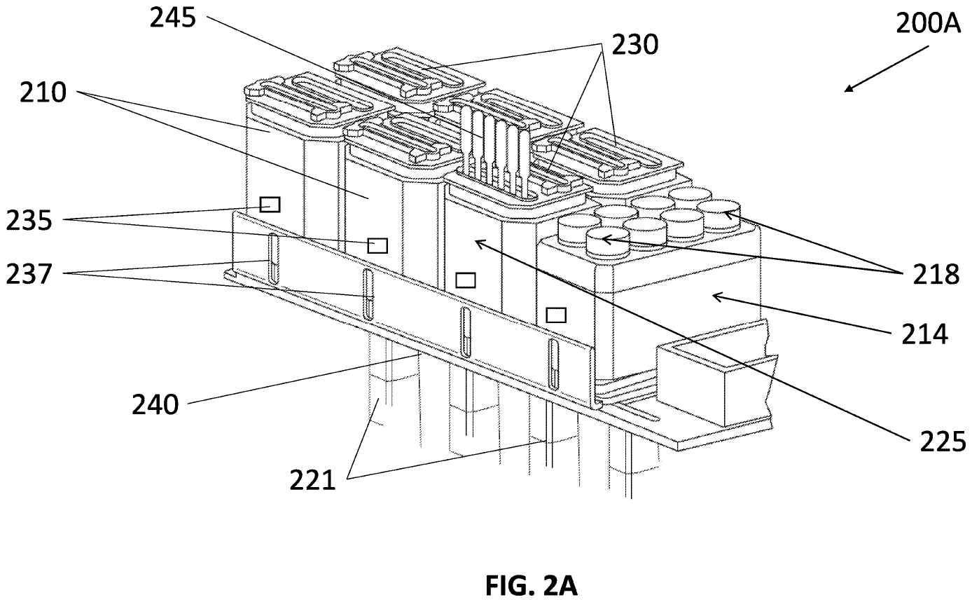

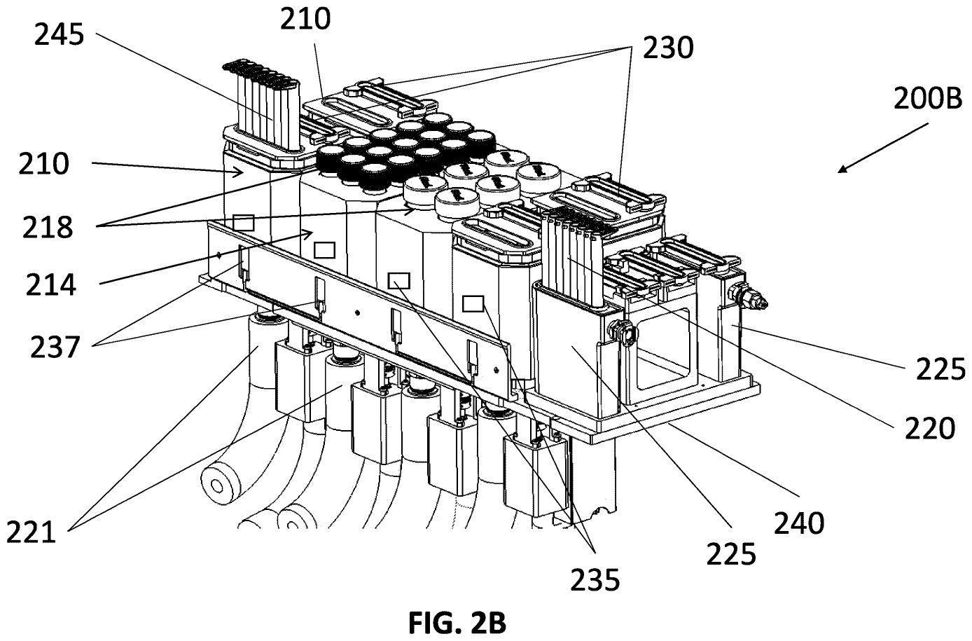

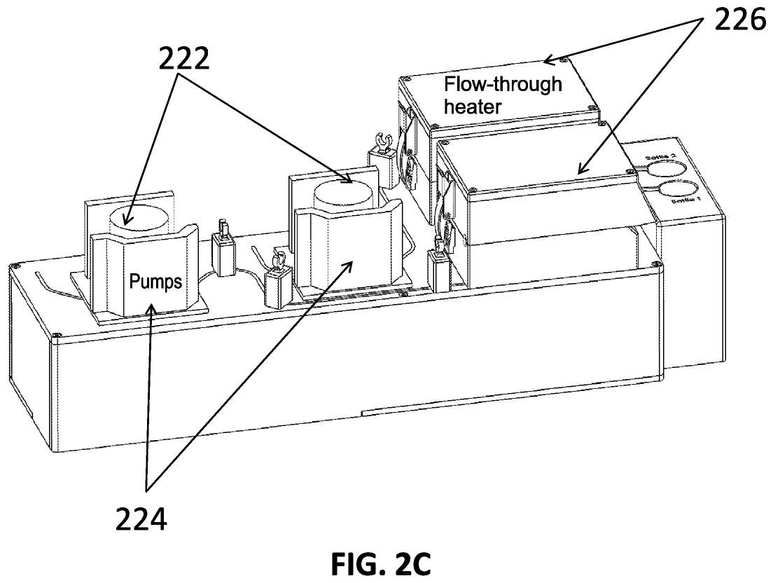

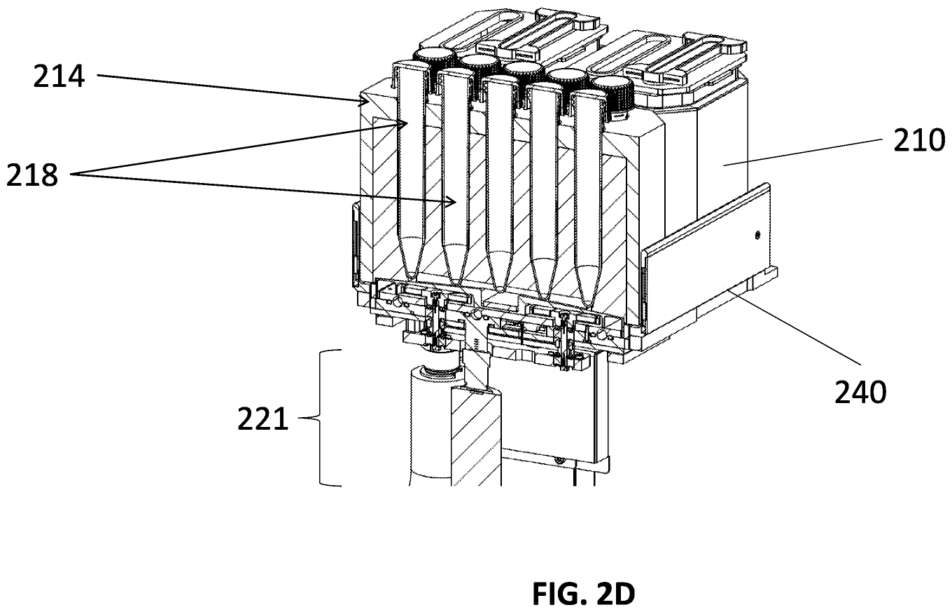

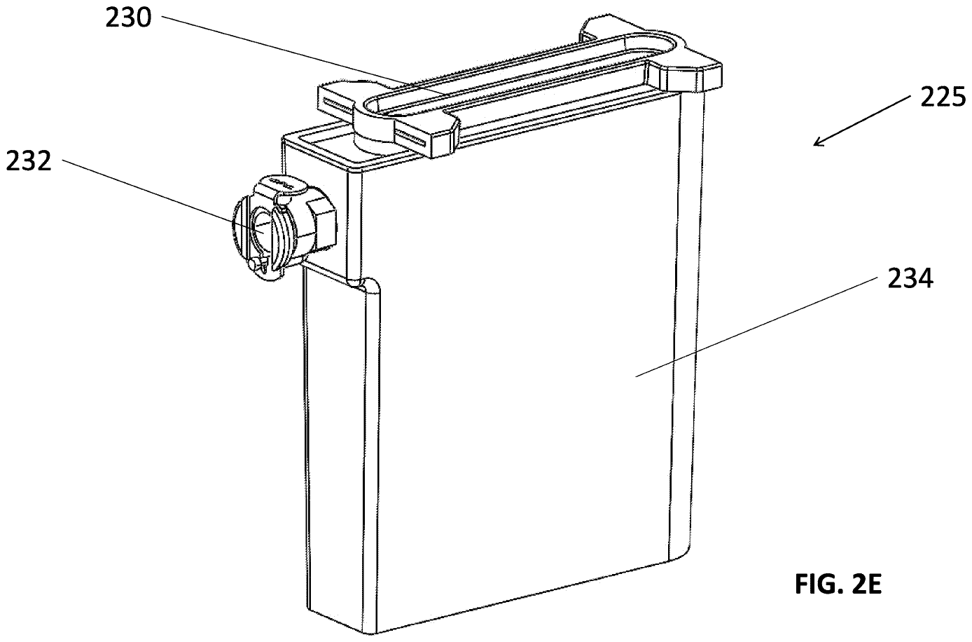

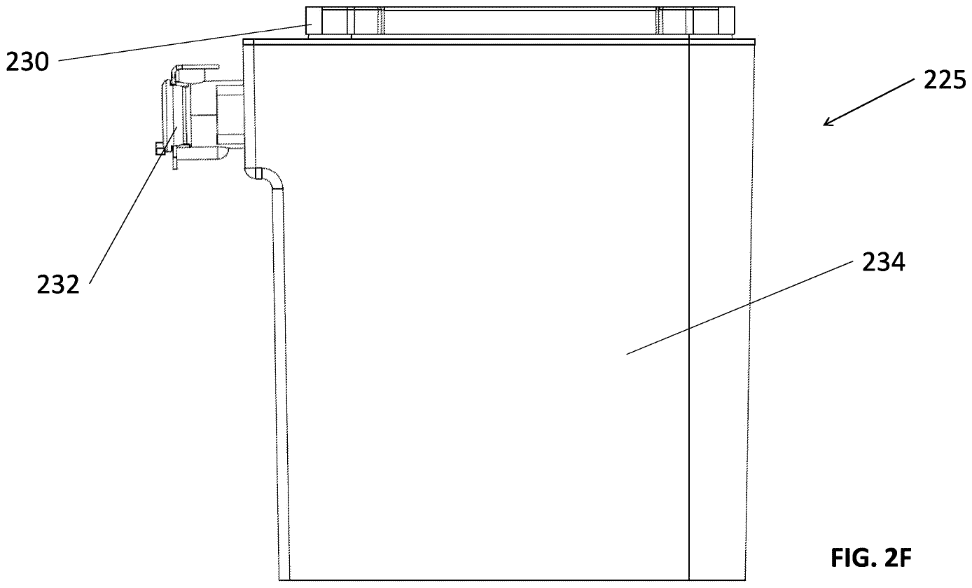

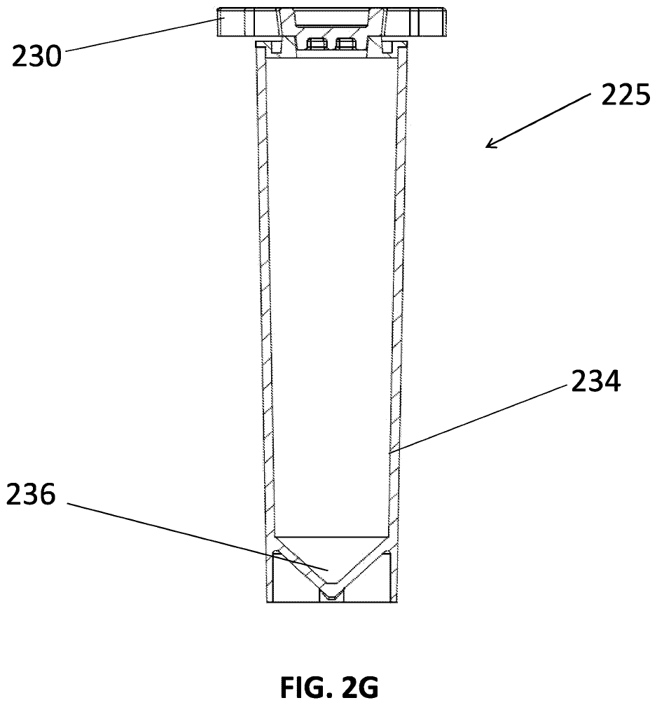

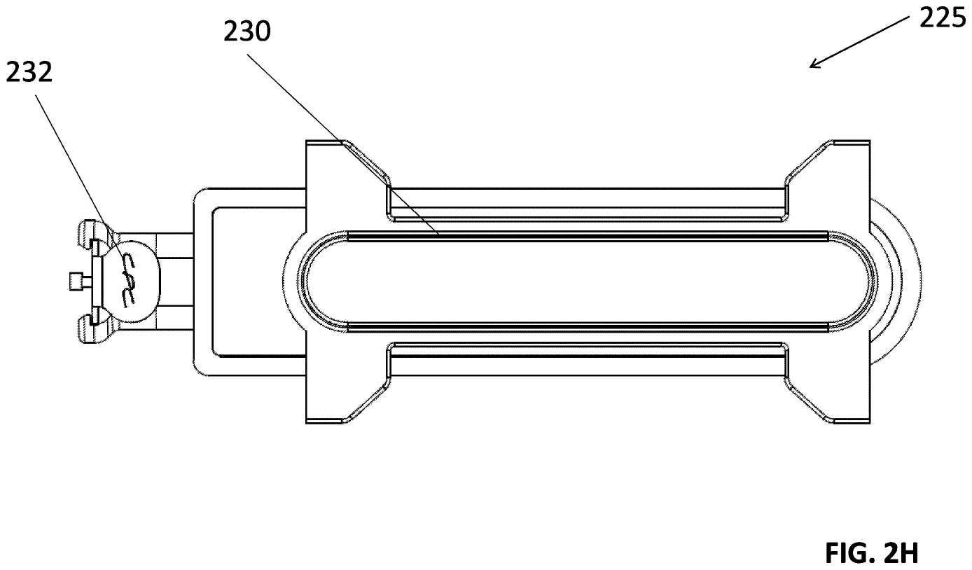

The small vessels 125 may be, e.g., smaller in size than the media troughs 110, and may be provided on the same support tray 140 of the automated cell culturing system 100A as the media troughs 110. The small vessels 125, which are elongated, may be coupled to reservoirs 122 via, e.g., a pump and a flow-through heater or cooler, as further discussed below with respect to FIG. 2C. The small vessels 125 may be arranged on the platform or support tray 140 similarly to the media troughs 110 but, e.g., may not be thermally insulated, unlike the media troughs 110. The reservoirs 122, which may have the shape of bottles, may be configured to supply a precise controlled amount of liquid or media to a small vessel 125, the amount being equal to the volume of the small vessels 125. For example, the reservoirs 122 may be configured to automatically supply the liquid or media to the small vessel 125 via the robotic arrangement 130. The volume of liquid transferred from the reservoirs 122 to the small vessel 125 may be controlled to correspond to the volume of the small vessel 125, so that any amount of liquid transferred from the small vessel 125 is precise and known. Accordingly, even if the liquid pump (discussed below with respect to FIG. 2C) is not precise enough to control the amount of liquid transferred from the reservoirs 122 to the small vessel 125, the fact that the volume of the small vessel 125 is small and known allows to precisely control the amount of transferred liquid. The pipettes 120 may be configured, under control of the robotic arrangement 130, to remove the same amount from of liquid or media the small vessel 125 and transfer it to its destination. The liquid or media transferred from the reservoirs 122 may be temperature controlled via a flow-through heater or cooling arrangement (not shown) further discussed in FIG. 2C. FIG. 1B illustrates an automated system 100B that also includes media troughs 110 and small vessels 125, similarly to FIG. 1A, but in a different configuration. For example, the number and location of each of the media troughs 110 and small vessels 125 may be different in the system 100B illustrated in FIG. 1B than in the system 100A illustrated in FIG. 1A. For example, on a given platform or tray 140, the number and combination of the media troughs 110 and the small vessels 125 may be configured as needed or required for a given application. The common feature between the various media receptables is that they are all arranged on the platform or tray 140, and that they can be automatically controlled and used to transfer media or liquid via the robotic arrangement 130. The remaining features such as, e.g., heating and cooling, stirring of the media or automation, may be similar between systems 100A and 100B.

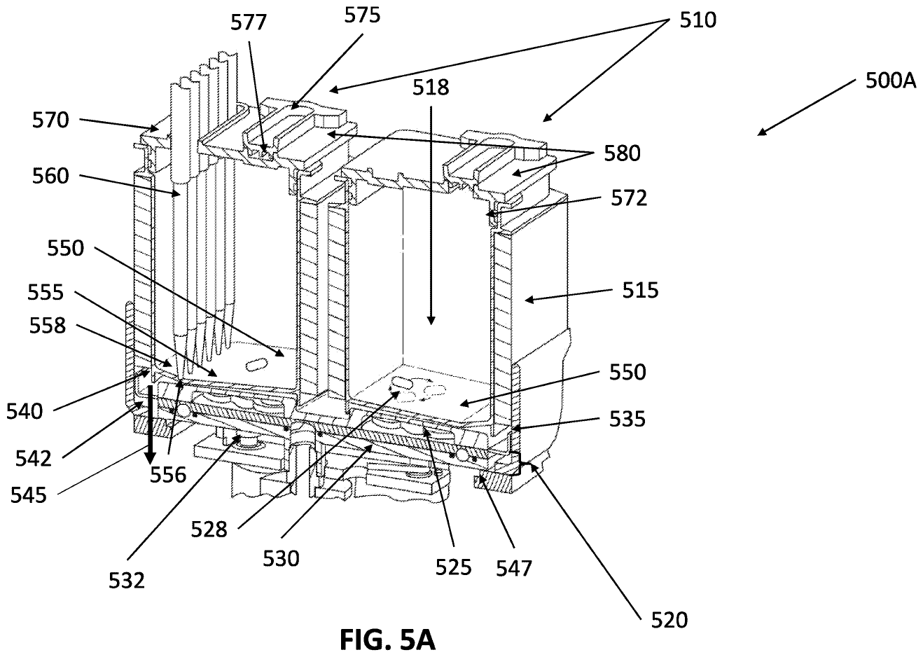

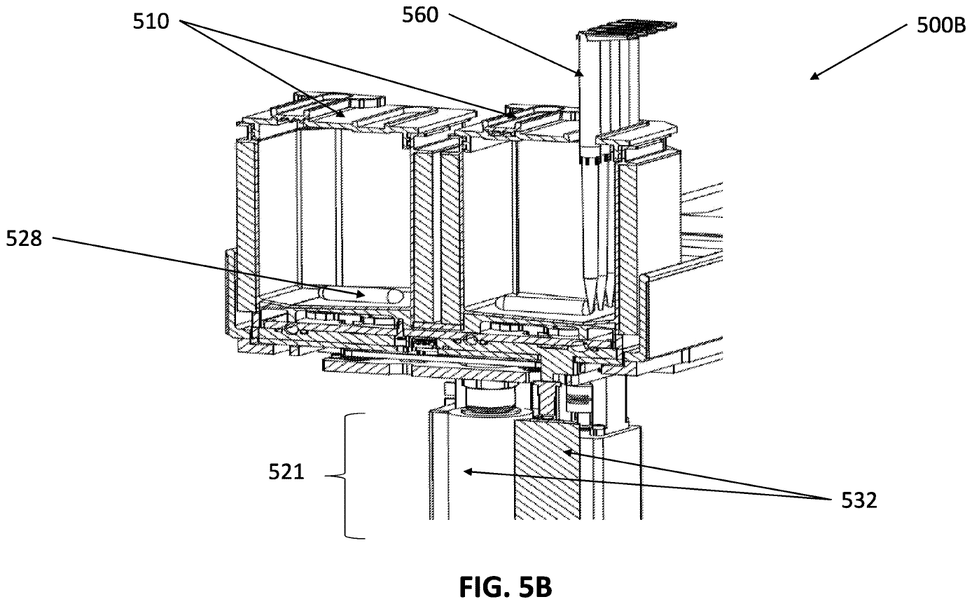

The automated cell culturing system 100A/100B thus includes one or more adapters 114 that include a plurality of capped centrifuge tubes 118, as described in greater detail with respect to FIG. 2D below. The adapters 114, also referred to as centrifuge tube adapters 114, may be positioned on the same platform or tray 140 as the media troughs 110 and may thus be subjected to, e.g., temperature control in the same manner as the media troughs 110, and may be stirred via magnetic stirrers controlled by the robotic arrangement. The adapters 114 may also be removed and replaced by one or more media troughs 110 or small vessels 125. Accordingly, the number of media troughs 110 that can be positioned on the platform or tray 140 may be increased to be up to eight media troughs 110 when the adapter(s) 114 are removed therefrom. On a given platform or tray 140, the number and combination of the media troughs 110, the small vessels 125, and the centrifuge tube adapters 114 may be configured as needed or required for a given application. The common feature between the various media receptables is that they are all arranged on the platform or tray 140, and that they can be automatically controlled and used to transfer media or liquid via the robotic arrangement 130. The centrifuge tubes 118 may be automatically controlled via the robotic arrangement 130 to be inserted in, or removed from, the adapters 114 so as to, e.g., transfer liquid in and out of the adapters 114. In an example, some of the media troughs 110 may be removed to allow the placement of centrifuge tube adapters 114. The support platform or tray 140 includes a plurality of stations (not shown and described below with respect to FIGS. 5A and 5B) underneath each media trough 110 and each centrifuge tube adapter 114, each station being configured to provide, e.g., temperature control or stirring of the liquid in the troughs 110 or adapter(s) 114. Alternatively, the support 140 may not include any station underneath the small vessels 125. Temperature control of the liquids transferred to the small vessels may be achieved via a flow-through heater and pump (not shown and described with respect to FIG. 2C).

FIGS. 2A-2H are illustrations of an automated media handling subsystem 200A/200B of an automated cell culturing system, in accordance with various examples of the disclosure. In FIG. 2A, the system 200A includes a plurality of container stations or media troughs 210 such as, e.g., up to eight media troughs 210 also referred to herein as container stations, as well as one or more centrifuge tube adapters 214 and one or more small vessels 225, on a platform or tray 240 that includes cooling, heating, and mixing arrangements underneath in stations 221. The adapters 214 are configured to hold a plurality of capped centrifuge tubes 218, and the small vessels 225 may be configured to receive pipettes 220. In examples, the cooling, heating, and mixing arrangements may be located below the tray 240 at the stations 221. Since the temperature control functionality is performed on the media troughs 210 and adapters 214 themselves via the stations underneath the supporting tray 240, the tray 240 may also include structures and materials configured to capture condensate that may develop on the troughs 210, but that could damage the other components of the automated system.

Each media trough 210 or centrifuge tube adapter 214 may include one or more external RFID tags or chips, bar codes, camera or other devices 235 to identify the media within the media trough 210 or adapter 214. The identification device or tags 235 may transmit the information via one or more antennas 237 to a receptor (not shown) on or near the tray 240. In examples, each media trough 210 and small vessel 225 includes a removable lid 230 on a top portion thereof, the removable lid 230 being configured to be manually or automatically removed under control of a controller (not shown) in order to allow storing or mixing of media or reagents therein. Some of the lids 230 are configured to enable access by a plurality of pipettes 220 simultaneously, for example, as manipulated by robotic arrangement such as, e.g., the robotic arrangement 130 illustrated in FIG. 1. For example, the pipettes 220 may be movable in and out of the trough 210 to enable access and removal of any media or reagent present therein, as further discussed with respect to FIGS. 5A and 5B below. The pipettes 220 may also be used to transfer liquid in and out of the centrifuge tubes 218 located in the centrifuge tube adapter 214.

Alternatively, the centrifuge tubes 218 may be transported in and out of the adapter 214 as needed to transfer the media or liquid therein. While the centrifuge tubes 218 are in the adapter 214, the media or liquid held therein may be subjected to, e.g., temperature control and/or stirring via a magnetic stirrer because the adapter 214 is arranged over a station 221. Underneath each media trough 210 and centrifuge tube adapter 214 is a station 221, each station 221 being configured to provide, e.g., temperature control or stirring of the liquid inside each media trough 210 and centrifuge tube adapter 214. The removable lid 230 may be made of, or include, a penetrable foil, to allow, e.g., shipment of the media or liquid when the media trough 210 is filled therewith. The support platform or tray 240 may thus include a plurality of stations 221 underneath each media trough 210 and each centrifuge tube adapter 214, each station 221 being configured to provide, e.g., temperature control or stirring of the liquid in the troughs 210 or adapters 214. The support or tray 240 may not include any station underneath the small vessels 225. Alternatively, a given station 221 may control two of the media troughs 210 or adapters 214 so that the given station 221 controls heating and stirring of two of the media troughs 210 or adapters 214, or of a pair including one media trough 210 and one adapter 214, which saves space under the tray 240 and saves power during operation of the temperature control and the stirring.

FIG. 2B illustrates an automated media handling subsystem 200B of an automated cell culturing system, in accordance with various examples of the disclosure. FIGS. 2A-2H are described concurrently and not every component described is depicted in every figure. In FIG. 2B, the system 200B includes media troughs 210, centrifuge tube adapters 214 configured to hold capped centrifuge tubes 218, and small vessels 225, the small vessels 225 may have pipettes 220 inserted therein, and the pipettes 220 may also be inserted in the media troughs 210 to manipulate or transfer the media or liquid held therein. FIG. 2B also illustrates the stations 221 arranged underneath the tray 240 and coupled thereto. For example, any given station 221 is configured to control two of the media troughs 210, adapters 214 and small vessels 225 in terms of temperature control and stirring control, and alternatively, each station 221 is configured to control one of the media troughs 210, adapters 214 and small vessels 225. Heating control may be accomplished electrically by the station delivering an amount of current to a heating coil arranged around the media troughs 210 or adapters 214. Cooling control may be accomplished by delivering an amount of cooling liquid through a fluid coil around the media troughs 210 and the adapters 214. Stirring of the media or liquid held in the media troughs 210 and adapters 214 may be performed by generating a rotating magnetic field in the station 221 to activate a magnetic stir (not shown but described below with respect to FIGS. 5A and 5B).

FIG. 2C illustrates the liquid supply dispensers 222, which are fluidly connected to, e.g., the reservoirs 122 illustrated in FIG. 1. In FIG. 2A, each of the liquid supply dispensers 222 may be coupled to a pump 224, and the pump 224 is configured to transfer the liquid media included in the dispensers 222 to one or more of the small vessels 225. Because the small vessels are not arranged on a temperature controlling station 221, unlike the media troughs 210 and the centrifuge tube adapters 214, temperature control inside the small vessels 225 may be performed via one or more flow-through heaters 226 configured to heat the media or liquids that are transferred from the dispensers 222 to the small vessels 225. FIG. 2D illustrates a centrifuge tube adapter 214 arranged on a tray 240, and the centrifuge tube adapter 214 shares the tray 240 with one or more media troughs 210. The centrifuge tube adapter 214 is arranged on a station 221 configured to provide, e.g., temperature control of the media or liquids inside each centrifuge tube 218.

FIGS. 2E and 2F illustrate a perspective view and a side view, respectively, of a small vessel 225, in accordance with examples of the current invention. The small vessel 225 includes a body 234, a removable lid 230 coupled to the body 234 and that can be removed to insert the pipettes 220 into the body 234. In examples, the small vessel 225 may also include an inlet port 232 configured to, e.g., introduce or remove a media or liquid into and out of the small vessel 225. FIGS. 2G and 2H illustrate a side view and a top view, respectively, of the small vessel 225, in accordance with examples of the current invention. In FIGS. 2G and 2H, the small vessel 225 has the removable lid 230 coupled to the body 234, and a floor 236 of the body 234 includes a non-flat portion configured to pool the liquid or media present inside the body 234 at a bottom portion thereof so as to facilitate extraction of the liquid or media from the body 234. For example, the non-flat floor 236 may include two pitched portions, wherein the pitch direction of each portion is opposite to the other so as to form a lowermost portion where the media or liquid held in the small vessel 225 may pool therein. In FIG. 2H, the small vessel 225 also includes inlet port 232 configured to, e.g., introduce a media or liquid into the small vessel 225. The removable lid 230 may be made of, or include, a penetrable foil, to allow, e.g., shipment of the media or liquid when the small vessel 225 is filled.

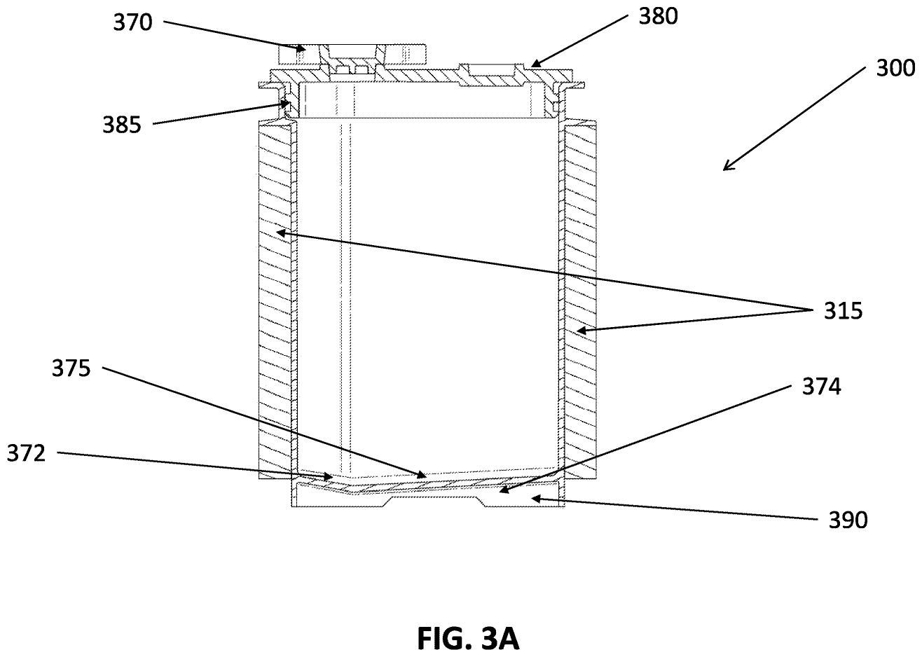

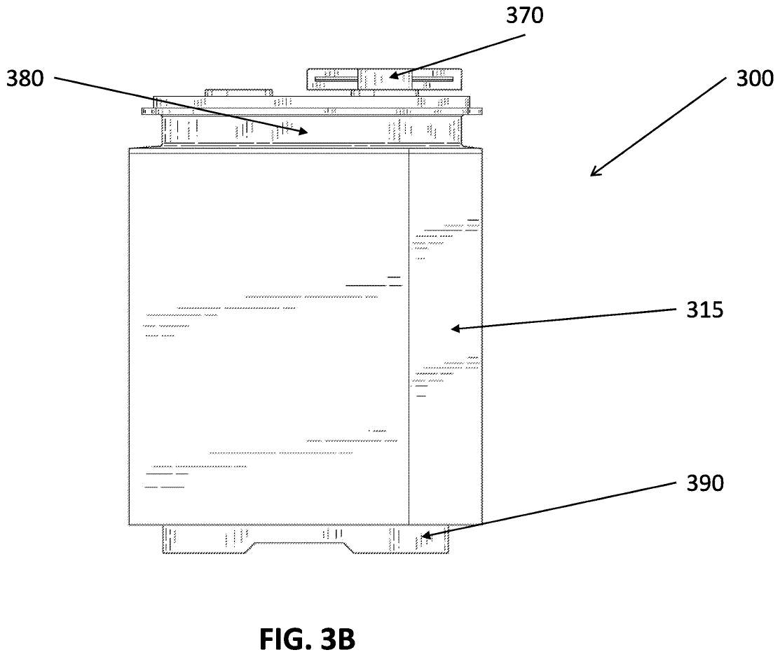

FIGS. 3A and 3B are side views of a media trough 300, according to various examples of the disclosure. In FIGS. 3A and 3B, media trough 300 includes walls 315 that may be insulative and/or autoclavable so as to sufficiently maintain any media and/or reagents present inside the troughs 300 at controlled temperatures. The walls 315 may also not be insulated and autoclavable, or may be insulated and not autoclavable. The walls 315 may be insulated by having a removable insulating sleeve affixed thereto. The insulated and/or autoclavable walls 315 may include RFID chips or tags such as the tags 235 illustrated in FIG. 2A. The media trough 300 may include a sealing lid 380 thereon, and a removable lid 370 configured to open and close, e.g., automatically under control of an automated system such as, e.g., the robotic arrangement 130 discussed above with respect to FIG. 1, to allow for the addition or removal of reagents or media into and out of the media trough 300. In various examples, the bottom surface of the sealing lid 380 includes a seal 385, the seal 385 being configured to seal to the opening of the media trough 300. For example, the seal 385 may be or include an O-ring gasket. The removable lid 370 may be arranged over a side of the media trough 300 and may be configured to receive, e.g., pipettes such as the pipettes 220 discussed above with respect to FIG. 2A, so as to avoid spillage during transfer of media or reagent in and out of the media trough 300.

The floor of an inside portion of the media trough 300 may be slanted with respect to the plane of the wall 315 and may include, e.g., two slanted floors 375 and 372. In examples, the slanted floors 375 and 372 may pitch towards each other, or may have slants that face each other, as illustrated in FIG. 3A. For example, the slanted floors 375 and 372, both pitching towards an area of the floor of the inside portion of the media trough 300, allow to reduce or eliminate having residual volume of media or reagent collecting at the bottom of the inside portion of the media trough 300, which may happen if the bottom of the inside portion of the media trough 300 is flat. The slanted floors 375 and 372 intersect at a line extending across a depth of the media trough 300, and the line may define a lowermost depth of the media trough 300. In addition, the area of the floor where slanted floors 372 and 375 intersect is vertically underneath the removable lid 370, so that when pipettes are inserted in the removable lid 370, the pipettes may access the entirety of the media or liquid present in the media trough 300. The trough 300 may include feet 390 configured to adjust a horizontal level of the trough 300 so that the slanted floors 372 and 375 inside the inside portion of the trough 300 remain tilted so as to reduce or eliminate any residual media or reagent held therein. In other examples, the feet 390 may be fixed at a set height, and may be thermally insulated by being covered with a thermal insulator, as illustrated in FIG. 3B, e.g., a removable thermal insulator. In various examples, the base 374 of the trough 300 may also have a slanted portion pitched at an angle with respect to a plane defined by wall 315.

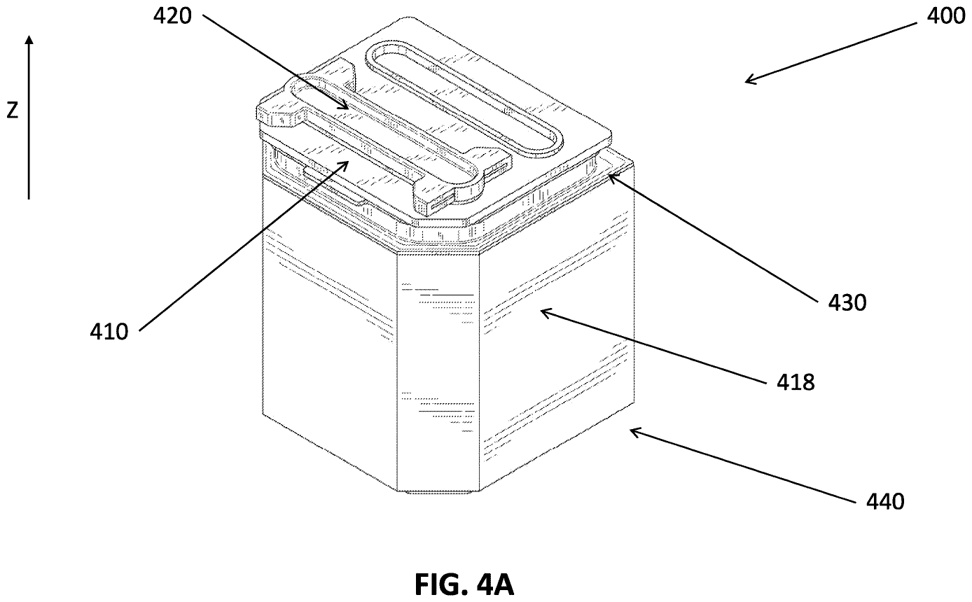

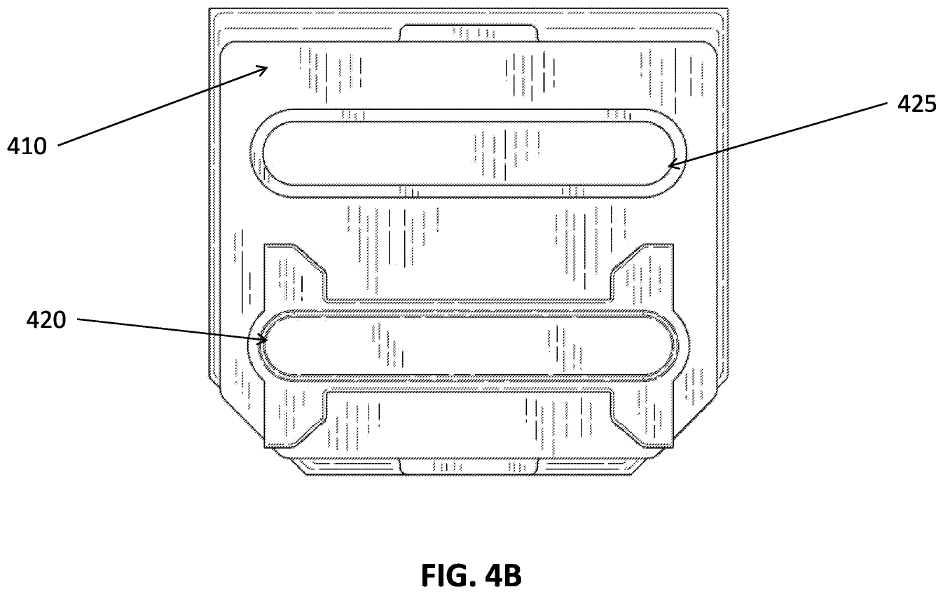

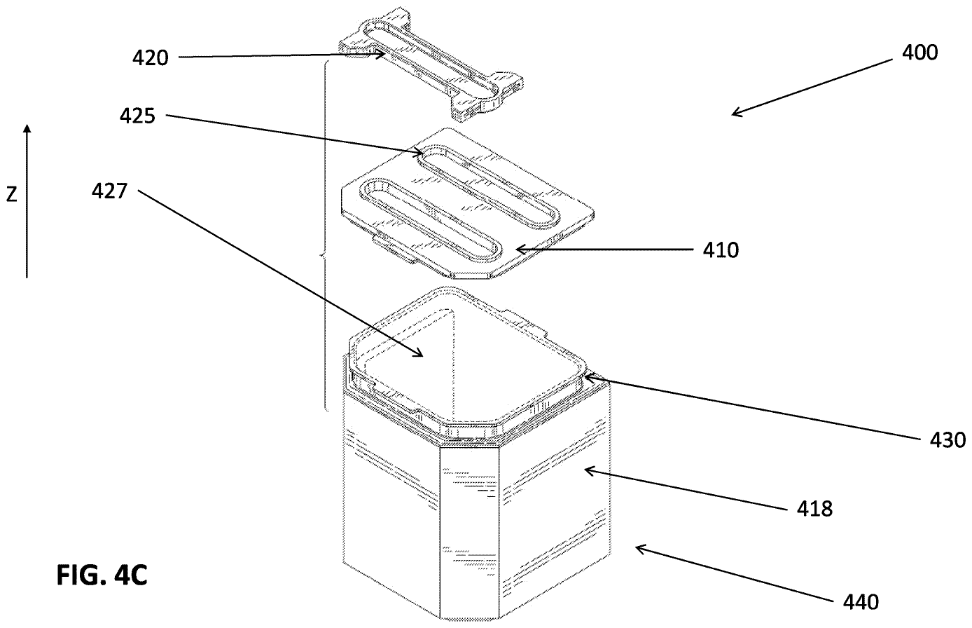

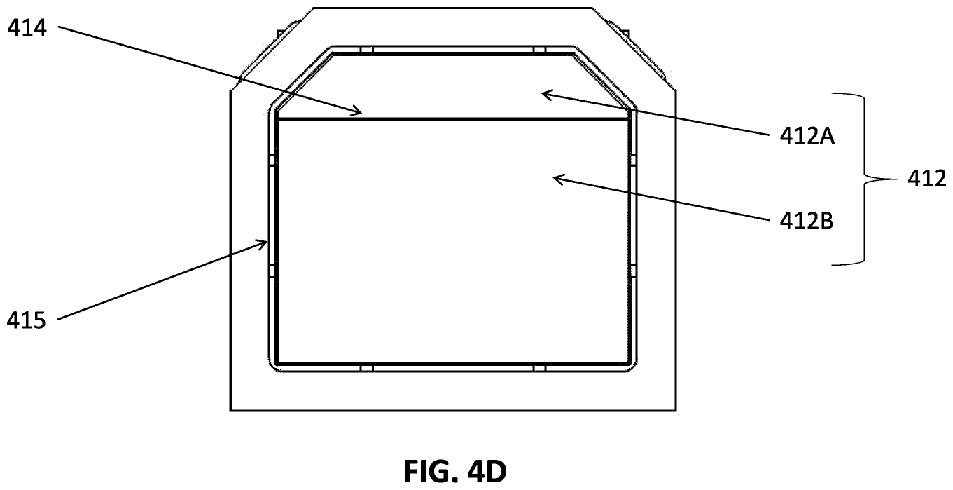

FIGS. 4A-4D illustrate perspective and exploded views of a media trough 400, in accordance with examples of the disclosure. FIGS. 4A-4D are described concurrently herein, and not every element discussed with respect to FIGS. 4A-4D is described in each of FIGS. 4A-4D. In examples, FIGS. 4A-4D illustrate a media trough 400 including a sealing lid 410 configured to sealingly cover the opening 427 of the media trough 400 by a sealing connection to a sealing rim 430 disposed over a perimeter of the opening 427. The media trough 400 may also include a body 440 which may be made of or include a metal, a rigid plastic, a combination thereof, or other like rigid and/or thermally insulating material. In further examples, the media trough 400 includes a removable access lid 420 that is configured to be removed from the sealing lid 410 in order to allow access to any media and/or reagent held inside the media trough 400 by, e.g., pipettes such as the pipettes 220 discussed above with respect to FIG. 2A. In other examples, the removable access lid 420 may be formed as a removable part of the sealing lid 410.

In an example, the rim 430 of the access opening 427 defines a rest, or detent, and the sealing lid 410 has a mating structure such as a flange or seal 415, illustrated in FIG. 4C, configured to sealingly fit with the rim 430 so as to ensure a substantially hermetic closing of the sealing lid 410 with the body 440 of the media trough 400. For example, the removable access lid 420 may have an elongated access opening axis, as illustrated in FIGS. 4A and 4B. In an example, the body 440 includes a thermally insulating material, which defines a lateral outermost extent thereof and cover an outer surface of the plurality of walls 418. For example, the thermally insulating material of the wall 418 may include polystyrene, fiberglass, polyurethane, cellulose, and the like.

FIG. 4B is a top view of the sealing lid 410 and the removable access lid 420. In various examples, the removable access lid 420 is configured to be removable, e.g., automatically removable via a robotic arrangement such as, e.g., the robotic arrangement 130 discussed above with respect to FIG. 1, in order to allow access to the media, liquid or reagent held in the media trough 400. In other examples, the sealing lid 410 includes a lid rest 425 on a surface thereof, the lid rest 425 being next to the removable access lid 420 and being configured to receive the access lid 420 when the access lid 420 is removed from the sealing lid 410 during, e.g., manipulation of the media and/or reagent held in the media trough 400. In various examples, the removable access lid 420 is configured to be removed substantially vertically from the access opening, i.e., in a direction along the “Z” axis illustrated in FIG. 4A. In other examples, the sealing lid 410 may also be removed in the vertical “Z” direction from the body 440 of the media trough 400. For example, a first removal force to remove the sealing lid 410 from the body 440 is substantially greater than a second removal force to remove the removable access lid 420 from the sealing lid 410. Accordingly, the removable access lid 420 may be removed without dislodging the sealing lid 410. FIG. 4C illustrates an exploded view of the media trough 400 showing the sealing lid 410 and the body 440 of the media trough 400. In examples, the sealing lid 410 includes the removable access lid 420 and the lid rest 425, and is configured to sealingly fit with the body 440 by a snug fit of the sealing lid 410 with the sealing rim 430 of the trough body 440.

FIG. 4D illustrates a view of the bottom floor 412 of the media trough 400, in accordance with various examples. The bottom floor 412 includes a flange or seal 415, the flange or seal 415 being configured to sealingly fit with a bottom portion of the body 440. The flange of seal 415 also defines a perimeter of an inside portion of the body 440. The floor 412 of the media trough 400 is double pitched, with one portion 412A pitching in one direction, and another portion 412B pitching in an opposite direction, and both portions 412A and 412B meeting along line 414 which marks the lowest point of the floor 412. With reference to FIGS. 4C and 4D, the vertical “Z” position of the line 414 is directly underneath the removable access lid 420 to facilitate efficient and complete removal of liquid or media present in the media trough 400.

FIGS. 5A and 5B are cross-sections of a media trough system, in accordance with various examples of the disclosure. FIGS. 5A and 5B are described concurrently and not every component described is depicted in every figure. In FIG. 5A, the media trough system 500A includes media troughs 510 supported by trays 520 and coupled to cooling/heating channels or cooling/heating elements 530 formed in the body of the trays 520, and which may be of, e.g., a laminated construction. In examples, the cooling/heating elements 530 may include openings at the ends thereof as cooling channels to provide chilled liquid cooling to the media and reagents present within the troughs 510, or may provide electrical heating to heat the media and reagents present within the troughs 510, and may reduce or eliminate the flow of cooling liquid to control cooling of the media and reagents present within the troughs 510. For example, the cooling/heating elements 530 include a hydronic channel. In other examples, cooling/heating elements 530 may be disposed within the body of the trays 520. In yet another example, hydronic, refrigerant-based, electric, or inductive heating and/or cooling elements may be utilized. The cooling/heating elements 530 may be controlled by a robotic arrangement such as robotic arrangement 130 described above with respect to FIGS. 1A and 1B, or by a computing device such as computing device 700 discussed below with respect to FIG. 7.

A rotating magnet 525 may selectively drive a ferrous stirring rod 528, located inside the body 518 of a media trough 510 in order to stir the media or reagent held inside the trough 510. The stirring rod 528 is activated via a motor 532 that controls the rotating magnet 525. The media trough system 500A may also include a perimeter condensate channel 540 that surrounds the media troughs 510 such that condensate that may form on the exterior of the troughs 510 may be collected in the channel 540, where it may be directed to a common drain 545. The channel 540 may be disposed adjacent an upper-most edge of the supporting surface of the floor 550 of the media trough 510. The media trough system 500A may also include a second condensate collection element 547, e.g., in the form of a removable tray, which may be disposed below the entire tray 520. The media trough system 500A may also include a moat 542 is adjacent a lower-most edge of the supporting surface of the floor 550 of the media trough 510, the moat 542 being coupled to a drain such as, e.g., drain 545. The tray 520 may define a perimeter at least partially surrounding the plurality of media troughs 510.

FIG. 5A also illustrates RFID sensors 535 embedded in each trough 510. Each trough 510 may have a plurality of insulated walls 515 on all sides thereof, the insulation being appropriate for, e.g., insertion into an autoclave for sterilization purposes. The walls 515 may also not be insulated and autoclavable, or may be insulated and not autoclavable. The walls 515 may be insulated by having a removable insulating sleeve affixed thereto. In examples, the troughs 510 may also have an inclined, or slanted, floor 550. For example, two adjacent troughs 510 may have slanted floors 550 that pitch away from each other. In some examples, two adjacent troughs 510 may have floors 550 that are coplanar with each other. In other examples, the floor 550 in each trough 510 may be a double pitched floor with two portions of the floor 550 being pitched in opposite directions to each other and intersect in an area of the floor 550 that is directly underneath the pipettes 560, as further discussed below. The floors 550 may be or include a durable surface that can resist damage during stirring of the media and/or the reagent held in the trough 510 by the stirring rod 528. In other examples, the walls 515 of the troughs 510 may be insulative and/or autoclavable walls 515 so as to maintain any media and/or reagents held inside the troughs 510 at controlled temperatures. In various examples, the troughs 510 have interior volumes 518 of, e.g., 400 ml, or in a range of, e.g., 100 ml-600 ml. In other examples, the troughs 510 may be disposable.

The floors 550 of the throughs 510 may include a first slanted portion 558 and a second slanted portion 555, the first and second slanted portions 558 and 555 being configured to avoid having media or reagent trapped at the bottom of the trough 510. For example, the slanted portions 558 and 555 may both be slanted towards a common portion or intersecting line 556 extending from the front of the floor 550 to the back thereof, thus forcing any media or reagent away from a corner of the trough 510 and into the common portion 556. In other examples, a plurality of pipettes 560 such as, e.g., eight (8) pipettes 560, may be insertable in the interior volume 518 of the media trough 510 in a location corresponding to the common area 556, the pipettes 560 being configured to remove, inject or otherwise manipulate media or reagent held inside the media trough 510. The group of pipettes 560 may be simultaneously inserted into the media trough 510 via opening 570 under control of, e.g., a user or robotic 130. For example, the opening 570 may be aligned above the lowest area 556 in the media trough 510.

In other examples, an elongated opening 570 may be formed inside the sealing lid 580 by removing a removable access lid 575, and the pipettes 560 may be inserted in the elongated opening 570. In examples, the elongated opening 570 may snugly fit the contour of the pipettes 560 so as to avoid spillage of media or reagent during transfer thereof in or out of the trough 510. In an example, upon being removed, the access lid 575 may be positioned on a lid rest or detente 577 next to the elongated opening 570 to prevent inadvertent dislodgment thereof while the media or reagent is being manipulated in the trough 510. In various examples, the two lids 575 and 580 are each secured to a corresponding trough 510 to prevent spillage of media or reagent from the trough 510.

In examples, both the lids 575 and 580 may be, e.g., robot-friendly and may be coupled to an automated system controller, such as the robotic controller 130 discussed above with respect to FIG. 1, configured to automatically open and close the lids 575 and 580 under control of a controller such as, e.g., the computing device 700 discussed below with respect to FIG. 7. One or more temperature sensors 572 may be added to the media trough 510, the temperature sensors 572 being coupled to a controller such as, e.g., the computing device 700 or the robotic arrangement 130. Accordingly, temperature regulation may be performed via, e.g., the computing device 700, where the temperature of the media or liquid present inside the trough 510 is sensed via the temperature sensor 572, and in response to the sensed temperature and the desired temperature, the controller (computing device 700) may control operation of the heating or cooling of the media trough 510 and any media or liquid held therein.

The lids 575 and 580 may have colors of varying opacity depending on the need to shield the media held in the trough 510 form outside light. The lids 575 and 580 may be transparent, may be opaque such as, e.g., black, or may have a milky or other color having a different opacity level. For example, the lids 575 and 580, as well as the insulated walls 515 of the media trough 510, may be opaque to light so as to shield the media or liquid held therein from light. In other examples, the lids 575 and 580, as well as the insulated walls 515 of the media trough 510, may be transparent to light so as to expose the media or liquid held therein to light.

In various examples, pairs of troughs 510 may be installed back-to-back on a single base or tray 520. The base or tray 520 may include a slanted upper surface to match the pitch of the bottom surface 550 of the troughs 510, as discussed above with respect to FIG. 3A. In an example, the base 520 may include a plurality of feet, such as feet 390 illustrated in FIG. 3A, such that the sides of the trough may be substantially vertical when the troughs 510 rest on the base 520. In examples, the base 520 may not be insulated, such that the media within the troughs 510 may be more easily heated and cooled. In other examples, the base 520 may be exposed, as illustrated in FIG. 5A.

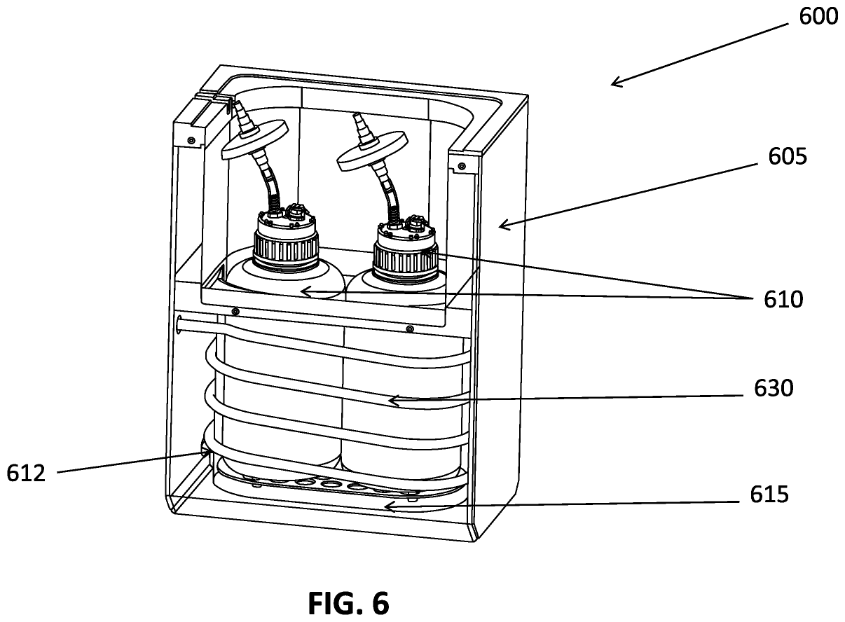

In FIG. 5B, the media trough system 500B includes similar features to the media trough system 500A discussed above. FIG. 5B further illustrates station 521 which includes one or more motors 532 configured to, e.g., control the stirring magnet 528 inside the media trough 510. For example, a single motor 532 may control the magnets of two of the media troughs 510. By having one station 521 control the stirring of two media troughs, it may be possible to save space underneath the media trough system 500B. The stations 521 may also control the temperature of the liquid or media held in a corresponding media trough 510 by, e.g., controlling the electrical heating of the liquid or media, or by controlling the amount of cooling liquid to be provided to the media trough 510 from liquid supply dispensers such as reservoirs 122 illustrated in FIG. 1B or the liquid supply apparatus 600 illustrated in FIG. 6 and further discussed below. In the examples illustrated in FIG. 5B, a single station 521 may control cooling, healing and stirring of two media troughs 510. In other examples, a single station 521 may control cooling, healing and stirring of more than two media troughs 510 and alternatively may control cooling, healing and stirring of a single media trough 521. By having a plurality of stations 521 under the media troughs 510, redundancy may be achieved, and different media may be controlled differently.

FIG. 6 illustrates a supply apparatus to an automated cell culturing system, in accordance with various examples of the disclosure. In examples, the liquid supply apparatus 600 is configured to supply a media or liquid at a controlled temperature to one or more small vessels such as, e.g., the small vessels 125 or 225 discussed above. The liquid supply apparatus 600 includes, e.g., one or more reservoirs 610, each reservoir 610 being configured to hold an amount of liquid such as, e.g., up to two liters of liquid. In examples, the liquid supply apparatus 600 includes a surrounding coil 630 which may be a tube coiled around the reservoirs 610, and which may circulate a cool or refrigerant liquid or gas to cool the liquid held inside the reservoirs 610. Alternatively, the surrounding coil 630 may circulate a warm or hot liquid or gas to heat the liquid held in the liquid supply apparatus 600, and another electric coil may be looped around the reservoirs 610 to electrically heat the liquid or media held therein. The liquid supply apparatus 600 further includes a sieve 615 on which to position the reservoirs 610. The liquid supply apparatus 600 may be encased in a non-cooled part such as, e.g., a rigid non-cooled part 605 which may include an insulative material. The insulative material of the part 605 may preserve the thermal integrity of the liquid held inside the reservoirs 610. As an additional example, the liquid supply apparatus 600 may include a liquid-level sensor 612 to monitor the level of liquid held inside the reservoirs 610.

FIG. 7 depicts a block diagram of a computing device that may be used as a controller for the media trough illustrated in, e.g., the figures above. In the illustrated example, the computing device 700 may include a bus 702 or other communication mechanism of similar function for communicating information, and at least one processing element 704 (collectively referred to as processing element 704) coupled with bus 702 for processing information. As will be appreciated by those skilled in the art, the processing element 704 may include a plurality of processing elements or cores, which may be packaged as a single processor or in a distributed arrangement. Furthermore, a plurality of virtual processing elements 704 may be included in the computing device 700 to provide the control or management operations for the cell culturing system described and illustrated above via a robotic arrangement such as, e.g., robotic arrangement 130 discussed above with respect to FIG. 1.

The computing device 700 may also include one or more volatile memory(ies) 706, which can for example include random access memory(ies) (RAM) or other dynamic memory component(s), coupled to one or more busses 702 for use by the at least one processing element 704. Computing device 700 may further include static, non-volatile memory(ies) 708, such as read only memory (ROM) or other static memory components, coupled to busses 702 for storing information and instructions for use by the at least one processing element 704. A storage component 710, such as a storage disk or storage memory, may be provided for storing information and instructions for use by the at least one processing element 704. As will be appreciated, the computing device 700 may include a distributed storage component 712, such as a networked disk or other storage resource available to the computing device 700. In examples, any of the volatile memory(ies) 706, the non-volatile memory(ies) 708, the storage component 710 and the distributed storage component may be referred to as a data repository.

The computing device 700 may be coupled to one or more displays 714 for displaying information to a user. Optional user input device(s) 716, such as a keyboard and/or touchscreen, may be coupled to Bus 702 for communicating information and command selections to the at least one processing element 704. An optional cursor control or graphical input device 718, such as a mouse, a trackball or cursor direction keys for communicating graphical user interface information and command selections to the at least one processing element. The computing device 700 may further include an input/output (I/O) component, such as a serial connection, digital connection, network connection, or other input/output component for allowing intercommunication with other computing components and the various components of, e.g., the cell culturing system and the robotic arrangement 130 discussed above with respect to FIG. 1.

In various examples, computing device 700 can be connected to one or more other computer systems via a network to form a networked system. Such networks can for example include one or more private networks or public networks, such as the Internet. In the networked system, one or more computer systems can store and serve the data to other computer systems. The one or more computer systems that store and serve the data can be referred to as servers or the cloud in a cloud computing scenario. The one or more computer systems can include one or more web servers, for example. The other computer systems that send and receive data to and from the servers or the cloud can be referred to as client or cloud devices, for example. Various operations of, e.g., the cell culturing system and the robotic arrangement 130 discussed above with respect to FIG. 1 may be supported by operation of the distributed computing systems.

The computing device 700 may be operative to control operation of the components of the media trough through a communication device such as, e.g., communication device 720, and to handle data generated by components of the media trough. In some examples, media or reagent handling data is provided by the computing device 700 in response to the at least one processing element 704 executing instructions contained in memory 706 or 708 and performing operations on data received from the media trough. Execution of instructions contained in memory 706 and/or 708 by the at least one processing element 704 can control operation of, e.g., the cell culturing system and the robotic arrangement 130 discussed above with respect to FIG. 1.

The term “computer-readable medium” as used herein refers to any media that participates in providing instructions to the processing element 704 for execution. Such a medium may take many forms, including but not limited to, non-volatile media, volatile media, and transmission media. Non-volatile media includes, for example, optical or magnetic disks, such as disk storage 710. Volatile media includes dynamic memory, such as memory 706. Transmission media includes coaxial cables, copper wire, and fiber optics, including the wires that include bus 702.

Common forms of computer-readable media or computer program products include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to the processing element 704 for execution. For example, the instructions may initially be carried on the magnetic disk of a remote computer. The remote computer can load the instructions into its dynamic memory and send the instructions over a telephone line using a modem. A modem local to computing device 700 can receive the data on the telephone line and use an infra-red transmitter to convert the data to an infra-red signal. An infra-red detector coupled to bus 702 can receive the data carried in the infra-red signal and place the data on bus 702. Bus 702 carries the data to memory 706, from which the processing element 704 retrieves and executes the instructions. The instructions received by memory 706 and/or memory 708 may optionally be stored on storage device 710 either before or after execution by the processing element 704.

### Selected Embodiments

Illustrative examples of the systems and methods described herein are provided below. An embodiment of the system or method described herein may include any one or more, and any combination of, the clauses described below.

Clause 1: A cell culture media container includes a body including a base and a plurality of walls extending from the base, wherein the plurality of walls together define a container opening; a sealing lid removably coupled to the body, the sealing lid including a removable access lid defined in an access opening of the sealing lid, the removable access lid being removably coupled to the sealing lid; and a seal disposed about a periphery of the sealing lid, wherein the seal is configured to removably secure the sealing lid to the container opening; wherein an interior floor of the base has two slanted floor portions, the slanted floor portions intersecting along a line located under the access opening in a direction perpendicular to a surface of the sealing lid.

Clause 2: The container of clause 1, wherein the body further includes an insulating material.

Clause 3: The container of clause 2, wherein the insulating material is disposed about the plurality of walls, defines a lateral outermost extent of the body and covers an outer surface of the plurality of walls.

Clause 4: The container of clause 3, wherein the base of the body is exposed.

Clause 5: The container of clause 3 or clause 4, wherein the insulating material includes one of polystyrene, fiberglass, polyurethane, and cellulose.

Clause 6: The container of any one of clauses 1-5, wherein the sealing lid is configured to be removed from the container opening in the direction perpendicular to a surface thereof; and the removable access lid is configured to be removed from the sealing lid in the direction perpendicular to the surface of the sealing lid therefrom at the access opening.

Clause 7: The container of clause 6, wherein a first removal force to remove the sealing lid from the body is greater than a second removal force to remove the removable access lid from the sealing lid.

Clause 8: The container of any one of clauses 1-7, wherein the seal includes an O-ring gasket.

Clause 9: The container of any one of clauses 1-8, wherein the base includes a first slanted portion pitched at a first angle to a plane defined by a first wall of the plurality of walls.

Clause 10: The container of clause 9, wherein the base further includes a second slanted portion pitched at a second angle to the plane defined by the first wall of the plurality of walls.

Clause 11: The container of clause 10, wherein the first angle is different than the second angle.

Clause 12: The container of any one of clauses 1-11, wherein the line defines a lowermost depth of an interior of the media container.

Clause 13: The container of any one of clauses 1-12, wherein the access opening is defined by an elongated access opening axis.

Clause 14: The container of clause 13, wherein the line is substantially parallel to the elongated access opening axis.

Clause 15: The container of any one of clauses 1-14, wherein the body includes an RFID sensor attached thereto.

Clause 16: The container of any one of clauses 1-15, wherein the sealing lid includes a detent configured to receive the removable access lid thereon.

Clause 17: The container of any one of clauses 1-16, wherein the body defines an opening, the opening including a rim and the sealing lid has a flange configured to snugly couple with the rim.

Clause 18: The container of any one of clauses 1-17, wherein the body includes one of metal, plastic, or a combination thereof.

Clause 19: The container of any one of clauses 1-18, wherein the plurality of walls are opaque to external light.

Clause 20: The container of any one of clauses 1-19, wherein the seal includes a penetrable material.

Clause 21: The container of clause 20, wherein the penetrable material includes a foil.

Clause 22: The container of any one of clauses 1-21, wherein the body includes one of a media trough, an adapter block for centrifuge tubes, and a vessel configure to hold a media volume corresponding to a volume of one or more pipettes.

Clause 23: An automated media handling system including a tray configured to arrange one or more cell culture media containers thereon; one or more stations underneath the tray, each of the one or more stations including a heating element disposed below the tray and thermally coupled to the one or more cell culture media containers arranged thereon; a cooling element disposed below the tray and thermally coupled to the one or more cell culture media containers arranged thereon; and a magnetic motor configured to rotatably actuate a stirring element in one or more of the cell culture media containers arranged thereon; a robotic arrangement configured to manipulate the cell culture media containers; and a controller configured to control operation of the cell culture media containers and of the one or more stations.

Clause 24: The system of clause 23, wherein a surface of the tray is at an angle to a horizontal plane.

Clause 25: The system of clause 23 or clause 24, wherein the one or more stations include a plurality of stations, and the tray further includes a channel disposed between adjacent stations.

Clause 26: The system of clause 25, wherein the channel is disposed adjacent an upper-most edge of each station.

Clause 27: The system of any one of clauses 23-26, further including a moat disposed adjacent a lower-most edge of each of station.

Clause 28: The system of clause 27, further including a drain fluidically coupled to the moat.

Clause 29: The system of any one of clauses 23-28, wherein the heating element includes an electric heating element.

Clause 30: The system of any one of clauses 23-29, wherein the cooling element includes a refrigerant cooling loop.

Clause 31: The tray of any one of clauses 23-30, wherein at least one of the heating element and the cooling element include a hydronic channel.

Clause 32: The system of any one of clauses 29-31, wherein the heating element includes the cooling element.

Clause 33: The system of any one of clauses 23-32, wherein the tray defines a perimeter wall at least partially surrounding the one or more stations.

Clause 34: The system of clause 33, wherein the perimeter wall defines an opening associated with each of the plurality of stations.

Clause 35: The system of any one of clauses 23-34, wherein the heating element and the cooling element of one or more stations are individually controllable.

Clause 36: The system of any one of clauses 23-35, wherein the heating element is disposed between the tray and the magnetic motor.

Clause 37: The system of any one of clauses 23-36, wherein the one or more stations includes up to eight stations.

Clause 38: The system of clause 37, wherein adjacent media containers have slanted floor surfaces that are pitched away from each other.

Clause 39: The system of clause 37 or claim 38, wherein every other media container arranged on the tray has a floor surface that are pitched in a same direction.

In accordance with various examples, instructions operative to be executed by a processing element to perform a method are stored on a computer-readable medium. The computer-readable medium can be a device that stores digital information. For example, a computer-readable medium includes a compact disc read-only memory (CD-ROM) as is known in the art for storing software or firmware. The computer-readable medium is accessed by a processor suitable for executing instructions configured to be executed.

This disclosure described some examples of the present technology with reference to the accompanying drawings, in which only some of the possible examples were shown. Other aspects can, however, be embodied in many different forms and should not be construed as limited to the examples set forth herein. Rather, these examples were provided so that this disclosure was thorough and complete and fully conveyed the scope of the possible examples to those skilled in the art.

Although specific examples were described herein, the scope of the technology is not limited to those specific examples. One skilled in the art will recognize other examples or improvements that are within the scope of the present technology. Therefore, the specific structure, acts, or media are disclosed only as illustrative examples. Examples according to the technology may also combine elements or components of those that are disclosed in general but not expressly exemplified in combination, unless otherwise stated herein. The scope of the technology is defined by the following claims and any equivalents therein.

The various embodiments described above are provided by way of illustration only and should not be construed to limit the claims attached hereto. Those skilled in the art will readily recognize various modifications and changes that may be made without following the example embodiments and applications illustrated and described herein, and without departing from the full scope of the following claims.

1. A cell culture media container comprising:
a body comprising a base and a plurality of walls extending from the base, wherein the plurality of walls together define a container opening;
a sealing lid removably coupled to the body, the sealing lid comprising:
a removable access lid defined in an access opening of the sealing lid, the removable access lid being removably coupled to the sealing lid; and
a seal disposed about a periphery of the sealing lid, wherein the seal is configured to removably secure the sealing lid to the container opening;
wherein an interior floor of the base has two slanted floor portions, the slanted floor portions intersecting along a line located under the access opening in a direction perpendicular to a surface of the sealing lid.

2. The container of claim 1, wherein the body further comprises an insulating material.

3. The container of claim 1 wherein the insulating material is disposed about the plurality of walls, defines a lateral outermost extent of the body and covers an outer surface of the plurality of walls.

4. (canceled)

5. (canceled)

6. The container of claim 1, wherein:
the sealing lid is configured to be removed from the container opening in the direction perpendicular to a surface thereof; and
the removable access lid is configured to be removed from the sealing lid in the direction perpendicular to the surface of the sealing lid therefrom at the access opening.

7. (canceled)

8. (canceled)

9. The container of claim 1, wherein the base comprises a first slanted portion pitched at a first angle to a plane defined by a first wall of the plurality of walls; and the base further comprises a second slanted portion pitched at a second angle to the plane defined by the first wall of the plurality of walls.

10. (canceled)

11. The container of claim 10, wherein the first angle is different than the second angle.

12. The container of claim 1, wherein the line defines a lowermost depth of an interior of the media container.

13. The container of claim 1, wherein the access opening is defined by an elongated access opening axis.

14. (canceled)

15. (canceled)

16. The container of claim 1, wherein the sealing lid comprises a detent configured to receive the removable access lid thereon.

17. The container of claim 1, wherein the body defines an opening, the opening comprising a rim and the sealing lid has a flange configured to snugly couple with the rim.

18. (canceled)

19. The container of claim 1, wherein the plurality of walls are opaque to external light.

20. The container of claim 1, wherein the seal comprises a penetrable material.

21. (canceled)

22. The container of claim 1, wherein the body comprises one of a media trough, an adapter block for centrifuge tubes, and a vessel configure to hold a media volume corresponding to a volume of one or more pipettes.

23. An automated media handling system comprising:
a tray configured to arrange one or more cell culture media containers thereon;
one or more stations underneath the tray, each of the one or more stations comprising:
a heating element disposed below the tray and thermally coupled to the one or more cell culture media containers arranged thereon;
a cooling element disposed below the tray and thermally coupled to the one or more cell culture media containers arranged thereon; and
a magnetic motor configured to rotatably actuate a stirring element in one or more of the cell culture media containers arranged thereon;
a robotic arrangement configured to manipulate the cell culture media containers; and
a controller configured to control operation of the cell culture media containers and of the one or more stations.

24. The system of claim 23, wherein a surface of the tray is at an angle to a horizontal plane.

25. The system of claim 23, wherein the one or more stations comprise a plurality of stations, and the tray further comprises a channel disposed between adjacent stations, and wherein the channel is disposed adjacent an uppermost edge of each station.

26. (canceled)

27. The system of claim 23, further comprising a moat disposed adjacent a lower-most edge of each of station, and further comprising a drain fluidically coupled to the moat.

28. (canceled)

29. (canceled)

30. The system of claim 23, wherein the cooling element comprises a refrigerant cooling loop.

31. The tray of claim 23, wherein at least one of the heating element and the cooling element comprise a hydronic channel.

32. (canceled)

33. The system of claim 23, wherein the tray defines a perimeter wall at least partially surrounding the one or more stations; and wherein the perimeter wall defines an opening associated with each of the plurality of stations.

34. (canceled)

35. (canceled)

36. (canceled)

37. (canceled)

38. (canceled)

39. (canceled)
